(19) United States
(12) Patent Application Publication
(10) Pub. No.: US 2024/0029899 A1
(43) Pub. Date: Jan. 25, 2024

(54) Cycle Thresholds in Machine Learning for Forecasting Infection Counts

(71) Applicant: Life Technologies Corporation, Carlsbad, CA (US)

(72) Inventors: Mahfuza Sharmin, Menlo Park, CA (US); Manimozhi Manivannan, Redwood City, CA (US); David Woo, Foster City, CA (US); Imran Mujawar, Goose Creek, SC (US); Manoj Gandhi, San Ramon, CA (US)

(73) Assignee: Life Technologies Corporation, Carlsbad, CA (US)

(21) Appl. No.: 18/225,065

(22) Filed: Jul. 21, 2023

Related U.S. Application Data
(60) Provisional application No. 63/391,740, filed on Jul. 23, 2022.

(51) Int. Cl. G16H 50/80; G16H 50/20; C12Q 1/686

(52) U.S. Cl. CPC G16H 50/80; G16H 50/20; C12Q 1/686

(57) ABSTRACT

Methods for forecasting case counts for a future date in one or more geographic areas of persons infected by a disease is disclosed. The presence of the disease in a biological sample is testable by a polymerase chain reaction (PCR) test. A load of one or more pathogens associated with the disease correlates with a PCR cycle which indicates presence of the one or more pathogens, and is referred to as a threshold cycle (Ct). Data relevant to forecasting the case counts including Ct data and other data is received. The Ct data comprises Ct values from PCR tests of biological samples from persons within the one or more geographic areas. Arrays of feature data for processing by a trained machine learning model are generated, comprising Ct features and other features obtained from the data. A forecasted number of infected persons are generated by processing the arrays using machine learning.

### CROSS-REFERENCE TO RELATED APPLICATIONS

This application claims the benefit of U.S. Provisional Application Ser. No. 63/391,740 filed on Jul. 23, 2022. To the extent permitted in applicable jurisdictions, the entire contents of this application are incorporated herein by reference.

### BACKGROUND

This disclosure relates generally to technology for forecasting case counts during a disease outbreak.

### SUMMARY

In managing disease outbreaks, predicting future case counts is an important tool. While nationwide case counts can be forecast reasonably well using relatively simple models applied to historical nationwide case count data, effective logistical planning at the local level requires being able to make better predictions of future case counts at a local geographic area. One recent effort to address this problem is the β-AR model described in the following paper, which is incorporated herein by reference in its entirety: Matthew Le, et al., Neural Relational Autoregression for High-Resolution COVID-19 Forecasting published by FB Data for Good, Oct. 1, 2020 (available at: https://ai.meta.com/research/publications/neural-relational-autoregression-for-high-resolution-covid-19-forecasting) (“13-AR paper”).

Polymerase Chain Reaction (PCR) tests are widely used for determining infection by a pathogen such as a specific virus or bacteria, or other pathogens such as fungi, protozoa, worms or prions. A PCR test performs thermal cycling on a biological sample. The cycling amplifies DNA corresponding to a target sequence if that sequence is present in the sample. If the target sequence can be detected by the PCR instrument prior to a given cycle (e.g., before cycle 38 of a 40 cycle assay), then the test can be considered “positive” for the corresponding person being infected by a virus corresponding to that sequence. However, the PCR test provides more information than simply whether a person is positive or negative. It also provides the cycle threshold (Ct) which is the PCR cycle at which the relevant sequence is first sufficiently amplified to be detected. Because the PCR process amplifies DNA, the cycle at which a sequence is first detectable is, on average, inversely proportional to the amount of a given DNA sequence initially present in a given sample volume. In other words, a small Ct value suggests a much higher amount of a given DNA sequence than does a high Ct value. This has been shown to correlate to viral load, i.e., the amount of virus in the infected person.

Although PCR tests provide Ct data, typically only the binary “positive” or “negative” result data (and not the Ct data) is used for predicting incidence and epidemic trajectory. Hay et al. have shown that because the Ct data, on average, correlates with viral load, it can improve incident rate estimates and epidemic growth reproductive rate estimates. See Hay et al., “Estimating epidemiologic dynamics from cross-sectional viral load distributions”, in Science 373, eabh0635 (2021) 16 Jul. 2021, incorporated herein by reference in its entirety (“Hay paper”).

However, neither the β-AR model nor other existing models have leveraged Ct data to improve the forecasting of future case counts.

Embodiments of the present disclosure provide methods, systems, and computer program products to improve high resolution case count forecasting by generating and using features derived from Ct data from PCR tests. Specifically, Ct data is used to generate Ct features to improve machine learning model performance on case count predictions.

Further details of these embodiments are more fully-disclosed herein and in Sharmin et al., “Cross-sectional Ct distributions from qPCR tests can provide an early warning signal for the spread of COVID-19 in communities,” medRxiv preprint doi: https://doi.org/10.1101/2023.01.12.23284489, posted Jan. 14, 2023, which is incorporated herein by reference in its entirety.

### BRIEF DESCRIPTION OF THE DRAWINGS

FIG. 1 illustrates a high-level view of a computerized system in accordance with an exemplary embodiment of the present disclosure.

FIG. 2 is a block architecture diagram of the case count forecasting system referenced in FIG. 1.

FIG. 3 is a flow diagram illustrating a method used to generate Ct features in accordance an embodiment of the present disclosure.

FIG. 4 illustrates an exemplary computer system configurable by a computer program product to carry out embodiments of the present disclosure.

While the disclosure is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

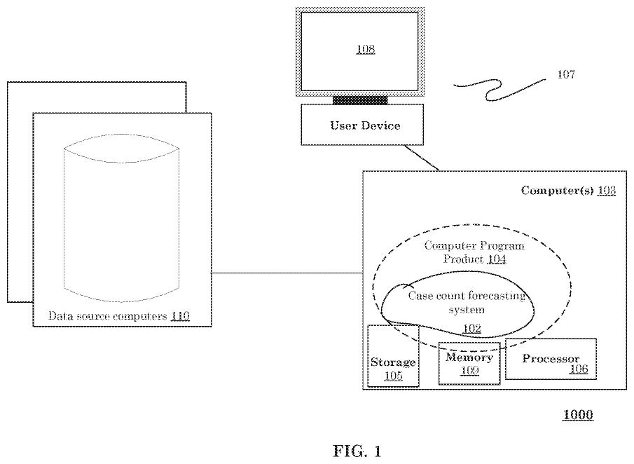

FIG. 1 illustrates System 1000 in accordance with an exemplary embodiment of the present disclosure. System 1000 comprises data source computers 101, one or more computers 103, and user device 107.

Instructions for implementing case count forecasting system 102 reside in computer program product 104 which is stored in storage 105 and those instructions are executable by processor 106. When processor 106 is executing the instructions of computer program product 104, the instructions, or a portion thereof, are typically loaded into working memory 109 from which the instructions are readily accessed by processor 106. In the illustrated embodiment, computer program product 104 is stored in storage 105 or another non-transitory computer readable medium (which may include being distributed across media on different devices and different locations). In alternative embodiments, the storage medium is transitory.

In one embodiment, processor 106 in fact comprises multiple processors which may comprise additional working memories (additional processors and memories not individually illustrated) including a graphics processing unit (GPU) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 106 may be implemented as one or more general purpose processors (preferably having multiple cores) without necessarily departing from the spirit and scope of the present invention.

User device 107 includes a display 108 for displaying results of processing carried out by case count forecasting system 102. Such a user device may include a mobile device such as a mobile phone, smart phone, smart watch, or tablet computer, and/or a laptop or desktop computer including a display 108. In some embodiments, alerts for impending epidemic waves in one or more community or communities of interest as detected by case count forecasting system 102 will be routed in real-time or near real-time to the one or more users via the user's respective user device 107. Such alerts may be displayed on user device 107 via app notifications to one or more mobile applications configured to receive results of processing carried out by case count forecasting system 102. Such app notifications may be displayed automatically on display 108 of user device 107, and may alert the user via audible sounds or vibrations.

In some embodiments, alerts for impending epidemic waves as detected by case count forecasting system 102 may be sent to the user via email alerts displayed on user device 107. In other embodiments, these alerts may be displayed on a user dashboard of case count forecasting system 102 that is shown display 108 of user device 107.

In a typical embodiment, data source computers 110 communicate with one or more of computers 103 over a computer network such as the Internet or another public or private network (not separately shown in FIG. 1) which may be a wide area or local network.

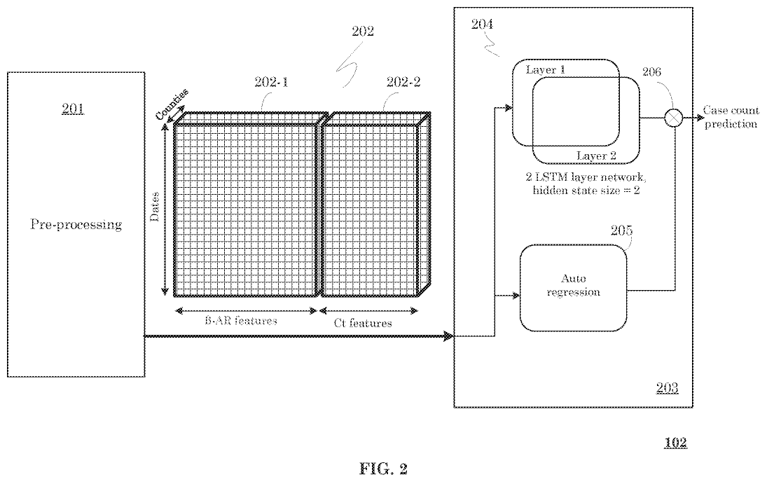

FIG. 2 is a high-level block architecture diagram of case count forecasting system 102 shown in FIG. 1 in accordance with an embodiment of the disclosure. System 102 comprises pre-processing block 201 and machine learning model 203. In the illustrated example, machine learning model 203 comprises a recurrent neural network (RNN) 204, auto regression model 205, and output multiplier 206. In the illustrated example, RNN 204 comprises two long term short term memory (LSTM) layers having a hidden state size of two. Output from auto regression model 205 and from RNN 204 are multiplied by output multiplier 206 which output case count prediction for one or more future dates within each of one or more geographic areas which, in this example, are counties.

In one example, operation of case count forecasting system 102 proceeds as follows. Pre-processing block 201 pre-processes cycle threshold (Ct) data and other data received from data source computers 110 shown in FIG. 1 and generates feature arrays 202 for input into machine learning model 203. Feature arrays 202 include three dimensional data that includes feature values and the corresponding geographic area (e.g., county, state, etc.) and dates associated with the data from which the feature values are derived.

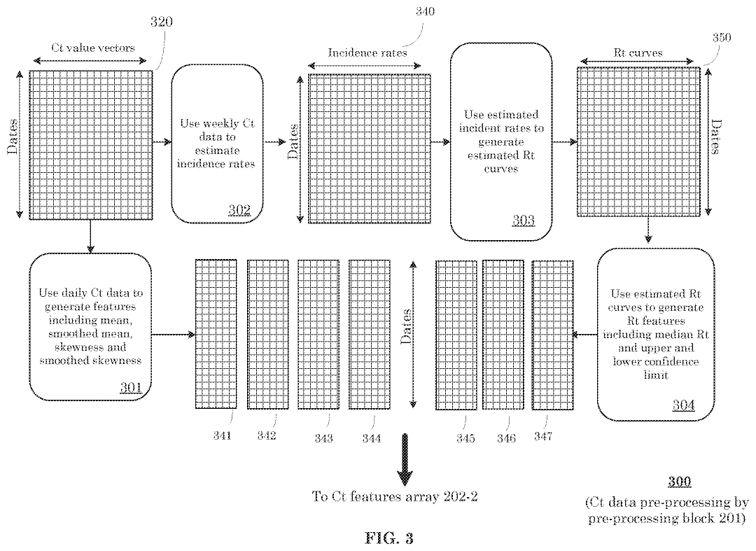

Feature data 202-2 includes Ct features, which are described in detail below in the context of FIG. 3.

Feature data 202-1 includes other features. In this example, the other features include features referenced in the β-AR paper referenced in the SUMMARY section above. Specifically, the β-AR features include features obtained from the following datasets: Confirmed Cases (New York Times collected data), Facebook Data for Good (FBDG) symptom survey, FBDG Movement Range Maps, Google Community Mobility data, doctor visits (CMU COVIDcast), Testing (COVID Tracking Project), and Weather (including average, minimum, maximum temperature and rainfall per county) (from NOAA GHCN). See β-AR paper at 6.

In this example, Ct feature data 202-2 and most of the β-AR feature data 202-1 are input into RNN 204 except that the β-AR Confirmed Cases feature is input into autoregression model 205.

In an alternative embodiments, additional features beyond those included in β-AR feature data 202-1 and Ct feature data 202-2 are used. For example, features related to disease variants are used in addition to β-AR features and Ct features. In one example, the time varying prevalence value of each of one or more of the top current variants are used as additional features. For example, in one embodiment, five variant features can be obtained for use by selecting the top five variants from GISAID, available, for example, at: https://www.gisaid.org/epiflu-applications/influenza-genomic-epidemiology/and the time varying prevalence values computed from the GISAID site for each of the five selected variants can be used as features.

In the illustrated example, machine learning model 203 comprises the neural relational autoregression model (β-AR model) described in the β-AR paper. However, in alternative embodiments, other machine learning models capable of generating case count forecasts from data that includes Ct data can be used. For example, the machine learning model 203 may also be updated over time, where machine learning model enhancements may be considered and incorporated into machine learning model 203.

FIG. 3 illustrates a method 300 used by pre-processing block 201 of FIG. 2 to generate Ct features 202-2. Method 300 operates on Ct data 320 to generate Ct features 341-347, which populate Ct features array 202-2, and are provided as part of feature array 202 to machine learning model 203 of FIG. 2.

Step 301 uses Ct data 320 to generate features 341, 342, 343, and 344 by determining, respectively, the mean, smoothed mean, skewness, and smoothed skewness of the vectors of Ct values. Specifically, respective sets of features 341-344 are computed for each respective date (e.g., each calendar day) that samples corresponding to respective Ct values were collected. And this is done for each of one or more geographic areas for which Ct data is provided (e.g., each county) (geographic area data dimension not separately shown in FIG. 3).

Features 341 and 343 (mean and skewness) are calculated based on all the Ct values collected for a given date within a given geographic area. Features 342 and 344 (smoothed mean and smoother skewness) are calculated based on a Ct values collected in a moving window of dates around the given date. In one example, the moving window is 14 days, meaning that, for example, the smoothed mean is based on the Ct values collected seven days prior and seven days after the given date. Furthermore, in one example, for each date in the rolling window, daily average Ct values are used for the smoothed mean and smoothed skewness determinations.

Step 302 uses weekly Ct data to estimate incidence rates and generate estimate incident rate data 340. In this example, an estimated incident rate is generated for each day in each county. In one example, this is done using the Gaussian process model from the Hay virosolver R-package using the recommended parameters. That package is available at https://jameshay218.github.io/virosolver/index.html and is incorporated herein by reference in its entirety.

Step 302 uses estimated incident rate data to generate estimated effective reproduction rate (Rt) curves. In one example, this is done by first computing a smoothed moving average of the estimated incident rates using a 14-day window. Then, the resulting smoothed incident rates are used to estimate Rt curves using EpiEstim available at https://cran.r-project.org/web/packages/EpiEstim/index.html and incorporated herein by reference in its entirety. Each estimated Rt curve is a time-series of estimated Rt values, for example, a series of daily estimated Rt values. In one example, additional data other than Ct-derived incidence estimates can also be submitted to EpiEstim to enrich the estimated Rt curve determinations. For example, case count data can also be submitted. In one example, this data can also be smoothed using, for example, a moving average calculation with a 14-day moving window. In one example, the EpiEstim recommended parameters of a mean serial interval of 6.14 and standard deviation of 3.96 can be used.

Step 304 then uses the estimated Rt curves to determine features 345, 346, and 347. Specifically, it determines a median estimated Rt value and upper and lower confidence limits for each day.

In some embodiments, the machine learning model performance will be automatically assessed over time, and features that show diminished utility will be excluded, and reconsidered if they appear to be of value again. In other embodiments, new features may be considered through test runs of machine learning model 203. If a new feature is determined to be of utility in forecasting case counts, such a new feature may be manually added to the machine learning model. The feature may be manually added using a user interface of case count forecasting system 102 in some embodiments.

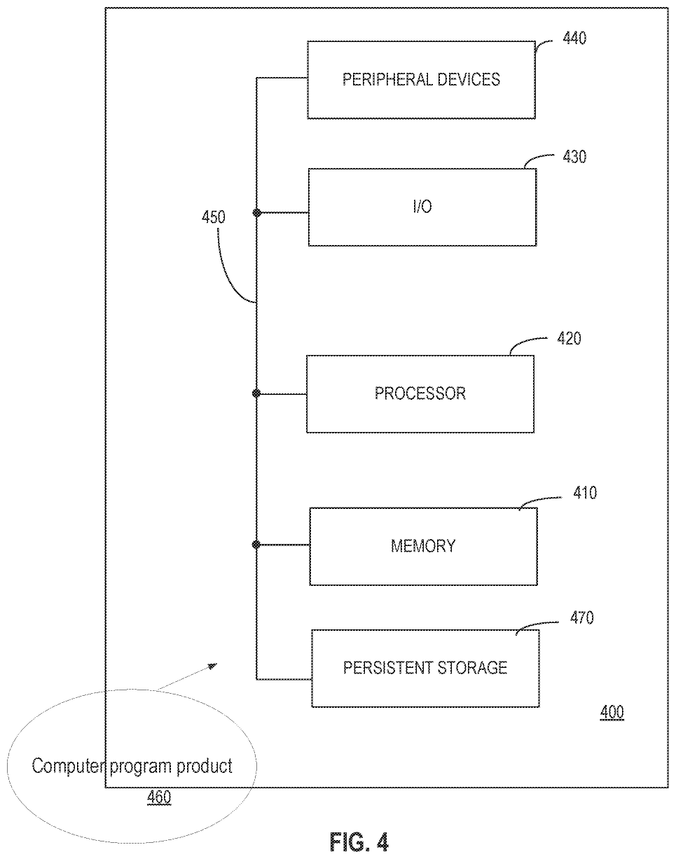

FIG. 4 illustrates an exemplary computer system configurable by a computer program product to carry out embodiments of the present invention.

In the example, computer system 400 may provide one or more of the components of an automated case count forecasting system configured to implement one or more logic modules and artificial neural networks and associated components for a computer-implemented case count forecasting system and associated interactive graphical user interface. Computer system 400 executes instruction code contained in a computer program product 460. Computer program product 460 comprises executable code in an electronically readable medium that may instruct one or more computers such as computer system 400 to perform processing that accomplishes the exemplary method steps performed by the embodiments referenced herein. The electronically readable medium may be any non-transitory medium that stores information electronically and may be accessed locally or remotely, for example, via a network connection. In alternative embodiments, the medium may be transitory. The medium may include a plurality of geographically dispersed media, each configured to store different parts of the executable code at different locations or at different times. The executable instruction code in an electronically readable medium directs the illustrated computer system 400 to carry out various exemplary tasks described herein. The executable code for directing the carrying out of tasks described herein would be typically realized in software. However, it will be appreciated by those skilled in the art that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 460 may reside in one or more storage persistent media (not separately shown) communicatively coupled to computer system 400 for loading and storage in persistent storage device 470 and/or memory 410 for execution by processor 420. Computer system 400 also includes I/O subsystem 430 and peripheral devices 440. I/O subsystem 430, peripheral devices 440, processor 420, memory 410, and persistent storage device 470 are coupled via bus 450. Like persistent storage device 470 and any other persistent storage that might contain computer program product 460, memory 410 is a non-transitory media (even if implemented as a typical volatile computer memory device). Moreover, those skilled in the art will appreciate that in addition to storing computer program product 460 for carrying out the processing described herein, memory 410 and/or persistent storage device 470 may be configured to store the various data elements referenced and illustrated herein.

Those skilled in the art will appreciate computer system 400 illustrates just one example of a system in which a computer program product in accordance with an embodiment of the present invention may be implemented. To cite but one example of an alternative embodiment, storage and execution of instructions contained in a computer program product such as, for example, computer program product 460, in accordance with an embodiment of the present disclosure may be distributed over multiple computers, such as, for example, over the computers of a distributed computing network.

What is claimed is:

1. A method, implemented by one or more computers, for forecasting case counts for a future date in one or more geographic areas of persons infected by a disease associated with one or more pathogens, the presence of which in a biological sample is testable by a polymerase chain reaction (PCR) test such that a load of the one or more pathogens typically correlates with a PCR cycle at which a PCR test of the biological sample indicates presence of the one or more pathogens, such a PCR cycle referred to as a threshold cycle (Ct), the method comprising:
receiving, at one or more computers, data relevant to forecasting the case counts, the data comprising Ct data and other data, the Ct data comprising Ct values from PCR tests of biological samples from persons within the one or more geographic areas;
generating, by the one or more computers, arrays of feature data for processing by a trained machine learning model implemented by the one or more computers, the feature data comprising Ct features obtained from the Ct data and other features obtained from the other data; and
processing, by the one or more computers, the arrays of feature data using the machine learning model to generate at least one forecasted case count comprising a forecasted number of infected persons for the future date in the one or more geographic areas.

2. The method of claim 1 wherein the Ct data comprises respective sets of Ct values from PCR tests conducted on respective dates, the PCR tests corresponding to persons in the one or more geographic areas.

3. The method of claim 2 wherein generating comprises determining a mean and a skewness of each of the respective sets of Ct values.

4. The method of claim 3 wherein generating further comprises determining a smoothed mean and a smoothed skewness of each of the respective sets of Ct values using Ct values from a rolling window of dates around a date of each respective set of Ct values.

5. The method of claim 2 wherein generating further comprises:
using the respective sets of Ct values to determine respective sets of estimated incident rates;
using the respective sets of estimated incident rates to determine respective sets of estimated effective reproductive rate (Rt) time series values; and
determining a mean and a skewness of each respective set of Rt time series values.

6. The method of claim 5 wherein generating further comprises:
determining a smoothed mean and a smoothed skewness of each respective set of Rt time series values.

7. The method of claim 1 wherein the machine learning model comprises a recurrent neural network.

8. The method of claim 7 wherein the machine learning model further comprises an autoregression model and an output multiplication function configured to multiply output of the recurrent neural network with output of the autoregression model to provide output of the machine learning model, wherein:
some features, including the Ct features, are processed by the recurrent neural network; and
at least one feature of the other features is processed by the autoregression model.

9. The method of claim 7 wherein the recurrent neural network comprises two long term short term memory (LSTM) layers.

10. The method of claim 9 wherein the two LSTM layers have a hidden state size of two.

11. The method of claim 2 wherein the respective dates corresponding to the respective sets of Ct data are dates on which a sample for a corresponding PCR test was collected.

12. The method of claim 1 wherein the one or more geographic areas comprises a plurality of respective geographic areas and further wherein the at least one case count comprises a plurality of respective case counts each corresponding to a different one of the respective geographic areas.

13. The method of claim 12 wherein the respective geographic areas are counties.

14. The method of claim 13 wherein using the respective sets of estimated incident rates to determine respective sets of estimated effective reproductive rate (Rt) time series values comprises using EpiEstim processing.

15. The method of claim 14 wherein using the respective sets of Ct values to determine respective sets of estimated incident rates comprises using Hay model processing.

16. The method of claim 1, further comprising providing a real-time or near real-time notification of the forecasted case count to a user device.

17. A computer program product comprising executable code stored in a non-transitory computer readable medium, the executable code being executable on one or more computer processors to execute the method of claim 1.

18. A non-transitory computer readable medium storing one or more executable instructions which when executed by at least one processor coupled to the non-transitory computer readable medium perform a method for forecasting case counts for a future date in one or more geographic areas of persons infected by a disease associated with one or more pathogens, the presence of which in a biological sample is testable by a polymerase chain reaction (PCR) test such that a load of the one or more pathogens typically correlates with a PCR cycle at which a PCR test of the biological sample indicates presence of the one or more pathogens, such a PCR cycle referred to as a threshold cycle (Ct), the method comprising:
receiving, at one or more computers, data relevant to forecasting the case counts, the data comprising Ct data and other data, the Ct data comprising Ct values from PCR tests of biological samples from persons within the one or more geographic areas;
generating, by the one or more computers, arrays of feature data for processing by a trained machine learning model implemented by the one or more computers, the feature data comprising Ct features obtained from the Ct data and other features obtained from the other data; and
processing, by the one or more computers, the arrays of feature data using the machine learning model to generate at least one forecasted case count comprising a forecasted number of infected persons for the future date in the one or more geographic areas.

19. A system for forecasting case counts for a future date in one or more geographic areas of persons infected by a disease associated with one or more pathogens, the presence of which in a biological sample is testable by a polymerase chain reaction (PCR) test such that a load of the one or more pathogens typically correlates with a PCR cycle at which a PCR test of the biological sample indicates presence of the one or more pathogens, such a PCR cycle referred to as a threshold cycle (Ct), the system comprising:
one or more processors configured for receiving data from one or more data source computers, the data relevant to forecasting the case counts, and comprising Ct data and other data, the Ct data comprising Ct values from PCR tests of biological samples from persons within the one or more geographic areas; and
one or more computer readable memories for storing a plurality of computer readable instructions, which upon execution by the one or more processors, perform the operations of:
generating arrays of feature data for processing by a trained machine learning model implemented by the one or more processors, the feature data comprising Ct features obtained from the Ct data and other features obtained from the other data; and
processing the arrays of feature data using the machine learning model to generate at least one forecasted case count comprising a forecasted number of infected persons for the future date in the one or more geographic areas.

20. The system of claim 19, wherein the plurality of computer readable instructions, upon execution by the one or more processors, further perform the step of providing a real-time or near real-time notification of the forecasted case count to a user device.
